(19) United States
(12) Patent Application Publication
(10) Pub. No.: US 2009/0306898 A1
(43) Pub. Date: Dec. 10, 2009

(54) Testing Particulate Materials

(75) Inventors: Donald A. Anschutz, Houston, TX (US); Allan R. Rickards, Tomball, TX (US)

Correspondence Address: STREETS & STEELE, 13831 NORTHWEST FREEWAY, SUITE 355, HOUSTON, TX, 77040, US

(73) Assignee: Prop Tester, Inc., Cypress, TX (US)

(21) Appl. No.: 12/133,264

(22) Filed: Jun. 4, 2008

(51) Int. Cl. G01V 9/00; G01N 33/00; G01N 15/08

(52) U.S. Cl. 702/11; 73/866; 73/38

(57) ABSTRACT

Embodiments include an apparatus and method for testing a particulate material suitable for use as a proppant. According to one embodiment, a sample of the particulate material is captured in the cavity of a test vessel between a cavity wall and a piston sealed with the cavity wall. A fluid is flowed into the test vessel from a fluid inlet of the test vessel to wet the sample of particulate material. The fluid is pressurized to a target fluid pressure greater than ambient pressure and heated to a target temperature greater than ambient temperature. The piston is moved into direct contact with the particulate material with sufficient force to crush at least a portion of the particulate material while maintaining one or both of the target temperature and the target pressure for one or more test cycles. Each test cycle has a duration of at least about 120 seconds and as long as about 24 hours.

### BACKGROUND OF THE INVENTION

1. Field of the Invention

The present invention relates to methods of testing particulate materials, and more particularly for testing proppants for use in downhole fracturing operations.

2. Description of the Related Art

Oil and natural gas are produced from wells having porous and permeable subterranean formations. The porosity of the formation permits the formation to store oil and gas, and the permeability of the formation permits the oil or gas fluid to move through the formation. Sometimes the permeability of the formation holding the gas or oil is insufficient for economic recovery of oil and gas. In other cases, during operation of the well, the permeability of the formation drops to such an extent that further recovery becomes uneconomical. In such circumstances, it is common to fracture the formation and prop the fracture in an open condition using a special-purpose particulate material referred to as a proppant. Fracturing is usually accomplished by hydraulic pressure using a gel-like fluid. The pressure is increased until cracks form in the underground rock. The proppants, which are suspended in this pressurized fluid, are forced into the cracks or fissures. When the hydraulic pressure is reduced, the proppant material prevents the formed fractures from closing again by “propping” the fractures open.

A wide variety of proppant materials are used, depending on the geological conditions. Typically, proppants are particulate materials, such as sand, glass beads, or ceramic pellets, which create a porous structure. Often, the proppants are coated with a resin to improve vital physical characteristics of the proppants. The oil or gas is able to flow through the interstices between the particles to collection regions, from which it is pumped to the surface. Over time, the pressure of the surrounding rock tends to crush the proppants. Fine particles referred to as “fines” may develop. Fines are particles smaller than the lowest screen size designated by regulations for a selected proppant. For example, for a selected proppant having a designated range of between 20 and 40 mesh (40 mesh being the smallest particle size in that range), fines are particles smaller than 40 mesh. The fines resulting from this disintegration tend to migrate and plug the interstitial flow passages in the propped structure. These migratory fines drastically reduce the permeability, lowering the conductivity of the oil or gas. Conductivity is a measure of the deliverability or the ease with which oil or gas can flow through the proppant structure and is important to the productivity of a well. When the conductivity drops below a certain level, the fracturing process is repeated or the well is abandoned.

The mechanical properties of a particular proppant material determine how effective that material is as a proppant and ultimately how much oil and gas will be produced from a well. For example, the particle size of a proppant has a significant impact on the permeability, and resulting ability for hydrocarbon flow through the fracture, of the proppant pack. Crush strength of the proppant is another vital physical characteristic of the proppant because the proppant is subjected to high pressure levels as they prop open the fracture. Early proppants were formed of materials such as sand, glass beads, walnut shells, and aluminum pellets. However, where closure pressures of the fracture exceed a few thousand pounds per square inch these materials are crushed resulting in a closure of the fracture. In response, proppants having high compressive strength have been designed to resist crushing under the high pressure levels experienced in use. The crush strength of the proppants is related to the composition and density of the proppant material. Another important physical characteristic of the proppant is the shape of the individual particle, wherein roundness and a high level of sphericity are important characteristics.

The importance of the physical characteristics of proppants is well recognized in the industry. The American Petroleum Institute (API) has issued Recommended Practices for proppant testing. For example, API Recommended Practices RP-56 covers testing procedures for sand used in hydraulic fracturing operations. RP-58 provides testing procedure for sand used in gravel packing operations. RP-60 provides testing procedures for high-strength proppants used in hydraulic fracturing operations. These Recommended Practices include testing procedures for determination of properties that include, inter alia, particle size, crush resistance and sphericity and roundness.

### SUMMARY OF THE INVENTION

Embodiments of the invention includes systems, methods, and software for testing particulate materials and evaluating the suitability of the particulate materials as proppants for downhole fracturing operations. For example, one embodiment provides a method of testing a particulate material. A sample of particulate material is captured in the cavity of a test vessel between a cavity wall and a piston sealed with the cavity wall. The sample of particulate material is heated to a target temperature greater than ambient temperature. A fluid is flowed through the sample of particulate material from a fluid inlet of the test vessel to a fluid outlet of the test vessel. The fluid flowing through the sample is pressurized to a target fluid pressure greater than ambient pressure. The piston is moved within the cavity into direct contact with the particulate material with a target level of force sufficient to crush at least a portion of the particulate material while maintaining one or both of the temperature and the fluid pressure for one or more test cycles.

Other embodiments of the invention and details thereof will be apparent from the following description and the appended claims.

### BRIEF DESCRIPTION OF THE DRAWINGS

FIG. 1 is a schematic diagram of an exemplary proppant testing system configured for testing a proppant under conditions of simultaneous heat, fluid, pressure, and a crushing level of force according to an embodiment of the invention.

FIG. 2 is a schematic diagram of the exemplary proppant testing system with the piston removed from the cavity of the vessel.

FIG. 3 is a schematic diagram of the exemplary proppant testing system wherein the proppant sample is simultaneously exposed to a crushing level of force, heat, fluid, and pressure.

FIG. 4 is a sectional view of a portion of the proppant sample after one or more testing cycles.

FIG. 5 is a graph of a particle size analysis performed on the proppant sample both before and after the hot wet crush test is performed.

FIG. 6 is a graph illustrating the correlation between particle size and baseline conductivity of a proppant at two-thousand psi for each of four different proppant materials.

FIG. 7 is a flowchart outlining a testing method according to an embodiment of the invention.

### DETAILED DESCRIPTION OF PREFERRED EMBODIMENTS

Embodiments of the invention include an apparatus and method for testing proppant materials under conditions that more closely replicate actual downhole conditions than do previously adopted industry testing procedures. According to one embodiment, a proppant sample is exposed to a direct crushing level of force, in combination with a simultaneous application of elevated fluid temperature, fluid flow, and static or dynamic fluid pressure. The proppant sample is first placed in the cavity of a crush cell, which includes a cylinder or other vessel having a fluid inlet and fluid outlet. A piston is placed in the cavity of the crush cell on top of the proppant sample, and the crush cell is placed in a hydraulic press. The hydraulic press moves the piston into direct contact with the proppant with sufficient force to crush at least some of the proppant particles. A liquid is passed into the cavity of the crush cell through the fluid inlet to wet the proppant. Once fluid flow has been established the fluid may continue in a dynamic flow regime or be shut in to simulate static conditions while holding back pressure on the device. The contents of the vessel (proppant and liquid) are heated while the fluid remains pressurized. A crushing level of force is added while heat, and fluid pressure are maintained for a period of time, and for one or more cycles. The proppant sample may be removed from the test vessel and a particle size analysis may be performed to determine how the particle size distribution has changed as a result of the combined heat, temperature, pressure, and crushing force. The change in particle size that results from testing the proppant under these conditions provides a more realistic indication of how the proppant is likely to perform under actual downhole conditions. Simulating the proppant pack in situ stress profile under conditions found in the field allows the user to select the optimum proppant of choice under their reservoir conditions.

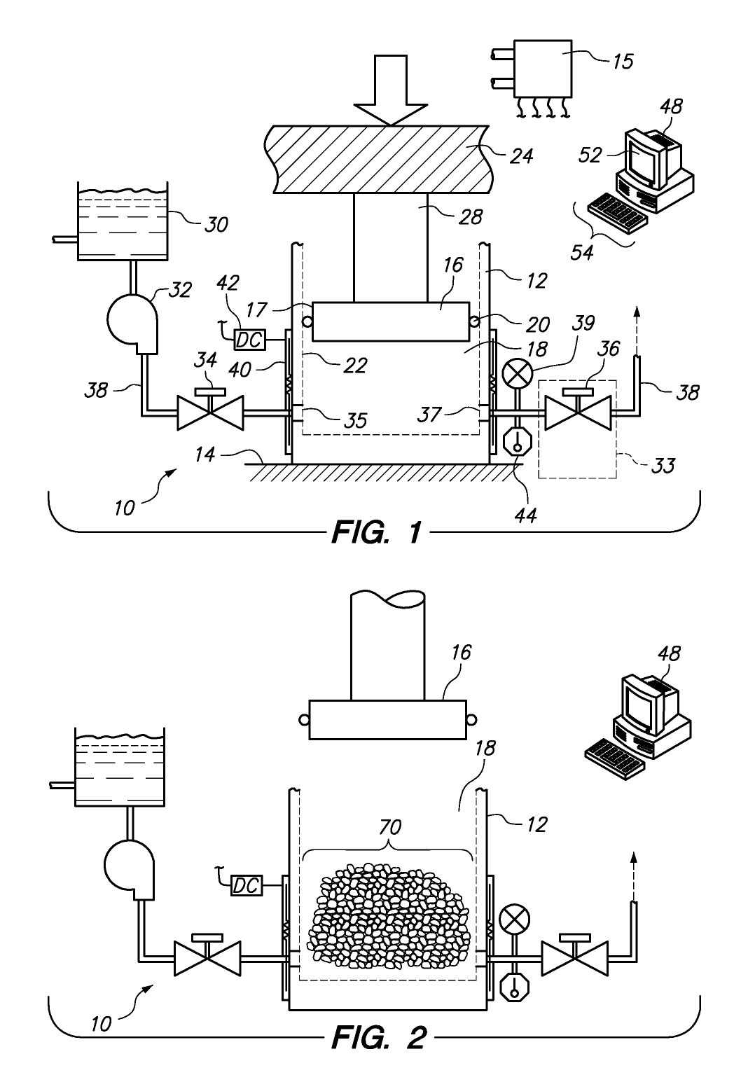

FIG. 1 is a schematic diagram of an exemplary proppant testing system 10 configured for testing a proppant under conditions of simultaneous heat, fluid pressure, and a crushing level of force according to one embodiment of the invention. The proppant testing system 10 includes a test vessel 12 supported on a platform 14 of a test fixture. The test fixture may include, for example, any of a variety of commercially available hydraulic presses. A piston 16 is movably disposed in a cavity 18 of the test vessel 12. A sealing member 20 seals between the piston 16 and a wall 22 of the cavity 18. The vessel wall 22 may be formed with any of a variety of cross-sectional shapes, with the perimeter 17 of the piston 16 matching the profile of the cavity wall 22. Typically, the vessel wall 22 will have a circular cross-section, in which case the piston 16 may have a generally cylindrical shape and the sealing member 20 may be an elastomeric “o-ring.” The test fixture also includes a hydraulically-powered crosshead 24 that may be moved up or down at a controlled rate by a controller 15. The crosshead 24 is engaged with a shaft 28 coupled to the piston 16. Moving the crosshead 24 up or down moves the piston 16 up or down to vary the volume bounded by the piston 16 and the cavity wall 22 and sealed by the sealing member 20.

The proppant testing system 10 further includes a fluid control system having a fluid source 30, a fluid pump 32, an inlet valve 34, and a back-pressure regulator 33 having an outlet valve 36. One example of a suitable back-pressure regulator 33 is a Tescom ER3000 computer-controlled back-pressure regulator, which may control pressure according to target fluid pressure values provided by a computer system 48. The inlet valve 34 is in fluid communication with an inlet port 35 of the test vessel 12 and the outlet valve 36 is in fluid communication with an outlet port 37 of the test vessel 12. Various segments of conduit 38 may be used to couple components of the fluid control system. The pump 32 may pump fluid from the fluid source 30, which may be a reservoir, through the inlet valve 34, into the sealed volume of the vessel cavity 18 through the inlet port 35. The back-pressure regulator 33 may control the pressure in the vessel cavity 18, such as by selectively restricting or completely closing fluid flow out of the outlet port 37, to achieve a desired fluid pressure in the vessel cavity 18 during testing. A pressure transducer 39 senses fluid pressure at the outlet port 37 and generates an electronic fluid pressure signal representative of the fluid pressure in response. The fluid pressure signal may be electronically transmitted to the back pressure regulator 33. Using the fluid pressure signal as feedback, the back pressure regulator may make adjustments as necessary to maintain a target pressure value provided by the computer 48. After testing, fluid may be selectively bled out of the vessel cavity 18 through the outlet port 37. If desired, the components of the fluid control system may be interconnected in a closed, filtered loop, so fluid exiting the cavity 18 is recirculated to the fluid source 30. Otherwise, the fluid exiting the cavity 18 may be discarded or returned to a fluid storage. If water is to be used as the fluid, the fluid source 30 may instead be water supplied by a utility company to the building that houses the proppant testing system 10, and the water exiting the outlet valve 36 may instead flow to a drain.

Water is a commonly available fluid that may be economically obtained for testing purposes. Water may also be present downhole either naturally or as a result of processes used during the exploration for or production of a hydrocarbon well, and is therefore especially suitable for simulating conditions in which water is likely to be present. Other examples of fluids commonly present downhole and which may be selected as the test fluid, include brine, hydrocarbon gas, hydrocarbon liquid, and hydrocarbon condensate. The selected fluid source 30 may include any of these fluids, either separately or in combination.

The proppant testing system 10 also includes a heater 40 in direct thermal contact with the test vessel 12 for heating the contents (e.g. fluid and proppant sample) of the test vessel 12. The heater 40 may be, for example, a commercially available 800-watt band heater secured to an outer perimeter of the test vessel 12 and tightened to ensure thermal contact with the test vessel 12. An AC power supply 42 may pass current through resistive heating element contained within the heater 40 to generate heat, which is transferred conductively to the test vessel 12 and contents thereof. A temperature sensor 44 is provided for sensing the temperature of fluid at the outlet port 37. The temperature sensor 44 generates an electronic signal representative of the sensed temperature. The temperature signal may be electronically transmitted to the heater 40, and the heater 40 may use the temperature signal as feedback to achieve and maintain a target temperature value provided by the computer 48.

The computer system 48 has software configured for coordinating tests to be performed on the proppant testing system 10. The computer system 48 provides a human interface to the proppant testing system 10, including a display 52 and input peripherals 54 such as a keyboard and pointing device. The input peripherals 54 may be used by personnel to set up and initiate tests to be performed on the proppant testing system 10, and to input target testing parameters for those tests, such as a target temperature, a target pressure, and a target force on the piston 16. The computer system 48 may be in electronic communication with the hydraulic press controller 15 included with the hydraulic press that controls movement of the crosshead 24. The computer 48 may also be in electronic communication with components of the fluid control system. For example, the computer may be electronically coupled to the pump 32 or a controller thereof, for selectively controlling power to the pump 32. The computer system 48 may also be electronically coupled to the inlet valve 34 or controller thereof, and to the back-pressure regulator 33. The computer 48 may be in electronic communication with the pressure transducer 39 for receiving the electronic fluid pressure signal. The computer system 48 may be electronically coupled to the heater 40 or a controller thereof, to control the amount of current passing through the heater 40 for achieving and maintaining a target temperature. While the computer system 48 may be configured to control elements of the proppant testing system 10 such as the valve 34, back-pressure regulator 33, crosshead 24, and heater 40, these elements may be additionally or alternatively controlled by separate controllers provided with these elements to enforce target fluid pressures, temperatures, and amount of crushing force requested by the computer system 48. In addition to receiving and displaying the target testing parameters, the computer may display actual values for the testing parameters such as position or rate of movement of the crosshead 24, detected fluid temperature and pressure, and cycle duration on the display 52. Personnel may monitor the actual testing parameters and target testing parameters on the display 52.

FIG. 2 is a schematic diagram of the exemplary proppant testing system 10 with the piston 16 removed from the cavity 18 of the vessel 12, such as by raising the crosshead 24 of FIG. 1. A proppant sample 70 to be tested is placed in the cavity 18 to create a proppant pack. The proppant sample may include, for example, ceramic particles, sand, glass beads, nut shells such as walnut shells that that have been treated or resin-coated, metal shot, metal particles, or combinations thereof. A variety of generally accepted sampling techniques known in the art may be used to obtain the proppant sample 70, such as may be prescribed by various standards bodies such as API and the International Organization for Standardization (ISO). The proppant sample may be obtained, for example, using “on-site” proppant materials, which are taken from a load of proppant to be used at a well fracturing site. A sampling device may be used to take the proppant sample from a proppant stream flowing from a conveyor belt onto a blender, truck, or rail car. The sampling device may be passed at a uniform rate from side to side through the full stream width of the proppant stream. A better proppant sample may be obtained by allowing the proppant to flow for at least two minutes after initial flow prior to taking the first proppant sample.

The proppant sample 70 to be tested need not be separated into a specified range of grain sizes prior to testing. Rather, the proppant sample 70 may contain the same particle size distribution as the proppant to be used at a well site. Typically, the proppant sample 70 may include particles with a range of grain sizes from about 200 μm to about 2000 μm. While the proppant sample 70 need not be separated, the particle size distribution of the proppant sample 70 may be obtained prior to testing the proppant sample 70 for use as a baseline. The particle size distribution may be obtained, for example, by passing the proppant sample 70 through a series of sieves having a progressively smaller mesh size, and then weighing the portion retained by each sieve to determine the percentage of that portion of the weight of the entire proppant sample. The separated portions may then be recombined before testing so that the same representative particle size distribution remains. Alternatively, a photo-optical particle size analyzer, such as the “Haver-CPA 3-2” offered by Haver & Boecker, or a laser particle size analyzer (LPSA) may be used to determine the baseline particle size distribution for the proppant sample 70.

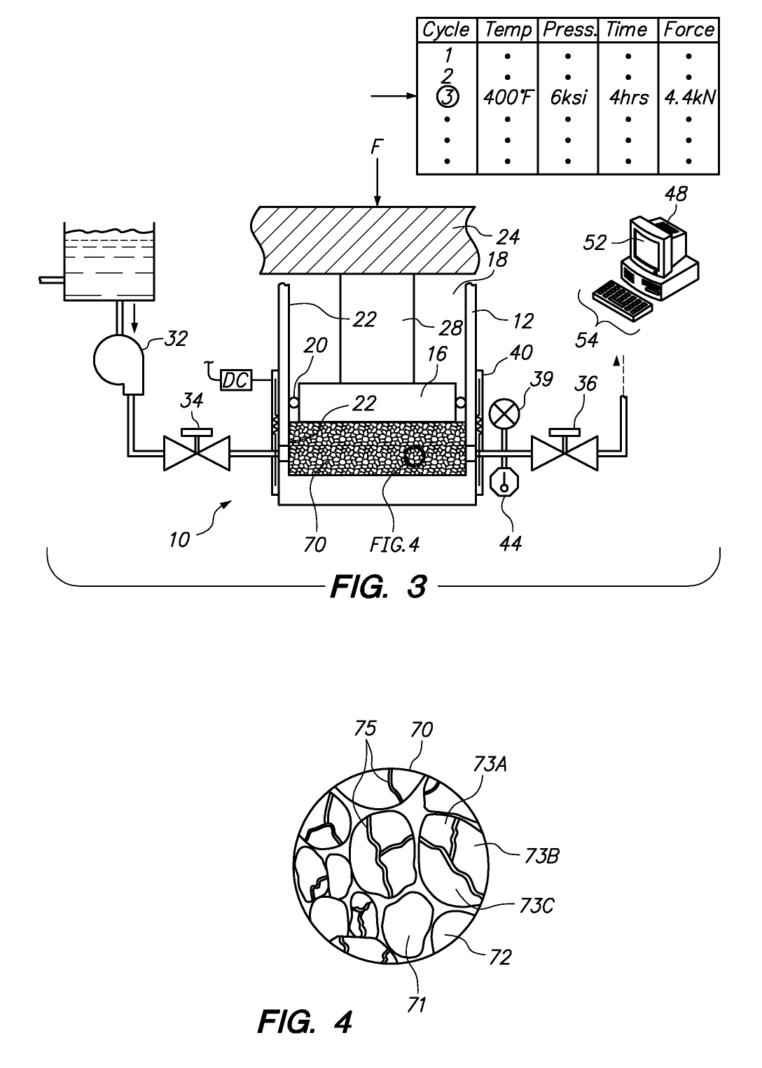

FIG. 3 is a schematic diagram of the exemplary proppant testing system 10 during performance of a “Hot Wet Crush” test according to an embodiment of the invention, wherein the proppant sample 70 is simultaneously exposed to a crushing level of force, along with heat and pressurized fluid flow. The piston 16 has been reinserted into the cavity 18 of the vessel 12 to capture the proppant sample 70 in the volume bounded by the piston 16 and the cavity wall 22 and sealed by the sealing member 20. Fluid flow is generated through this sealed volume and the proppant sample 70 captured therein by powering on the pump 32 and opening the valves 34, 36. The valve 34 may be moved fully open to minimize the restriction to flow into the vessel 12. The valve 36 may be partially closed to generate a flow restriction at the outlet port 37 to provide a controllable amount of back pressure on the fluid in the vessel 12. The computer system 48 may maintain a prescribed fluid pressure by monitoring the signal from the pressure meter 39 and varying the outlet valve 36 in response. The proppant sample 70 and the fluid in the vessel 12 may be heated by powering on the heater 40. The computer system 48 may monitor the signal from the temperature sensor 44 and vary the amount of current provided by the AC power supply 42 using solid-state relays, to maintain a target temperature. While maintaining the prescribed temperature and fluid pressure, the crosshead 24 is moved downward into direct contact with the proppant sample 70 with sufficient force to crush some of the particles. This simultaneous crushing force, fluid flow, and heat, and the subsequent analysis below, determines the actual crush strength of proppant under realistic well conditions.

An exemplary graphical user interface (“GUI”) 60 is shown as it may be displayed on a display 52 of the computer 48. The illustrated GUI 60 displays testing parameters such as the cycle number currently being performed, fluid temperature, fluid pressure, cycle time elapsed, and amount of force or stress imposed on the proppant sample 70 at the piston 16. Additional display information may include, for example, target values for testing parameters, such as the number of cycles to be performed, the target duration of each cycle, the target fluid pressure, and the target temperature.

The testing parameters, such as temperature, fluid pressure, level of crushing force, cycle duration, and number of cycles, may be programmed into the testing software on the computer system 48. Alternatively, personnel may manually input the testing parameters using the input peripherals 54. The testing parameters may be determined in a variety of ways. For example, a standards body may promulgate a set of testing parameters for the hot, wet crush test. The testing software may include these promulgated testing parameters. If these standards are periodically revised by the standards body, the software may be updated accordingly. Alternatively, the testing parameters may be selected according to site-specific parameters. For example, testing personnel may select the testing parameters according to the observed or anticipated range of heat and fluid pressure for a particular well site. The type of proppant material used in the proppant sample 70 may also be selected according to the type of proppant desired to be used at the site.

A producing hydrocarbon well will typically be exposed to wide variations in pressure. For example, during shut-in, fluid pressure may be about two-thousand pounds per square inch (psi), and an increased pressure of ten-thousand psi when flow is resumed. To simulate such variability in downhole conditions in the laboratory, fluid pressure and temperature may be varied during each testing cycle. For example, within a particular testing cycle, a dynamic fluid pressure may be imposed by selectively varying the back pressure in the cavity 18. The dynamic fluid pressure simulates the fluid pressure fluctuations that occur when a well is periodically shut in. Alternatively, during one or more cycles, a constant pressure may be imposed and maintained in the test vessel for a target time interval.

An elevated temperature may also be imposed on the proppant sample 70 to simulate the elevated temperatures typically present downhole. Elevated temperatures in the range of about 80 to 500 degrees Fahrenheit (26.7 to 260 Celsius) are suitable for testing. More typically, temperatures in the range of about 200 to 450 degrees Fahrenheit (93.3 to 232 Celsius) may be imposed. Even with water being used as the testing fluid, temperatures in excess of 212 degrees Fahrenheit (100 Celsius) may be imposed on the proppant sample 70 by virtue of the water being contained and pressurized within the sealed volume between the cavity wall 22 and the piston 16. Whereas water boils at 100 Celsius under atmospheric conditions, the elevated fluid pressure induced within the cavity 18 between the piston 16 and the cavity wall 22 allows the temperature to also be increased above 100 Celsius. The ability to increase temperature above the atmospheric boiling point facilitates simulating temperatures that can occur within a pressurized formation.

One or more testing cycles may be performed with the proppant sample 70 without removing the proppant sample 70 from the vessel 12. For example, in one cycle, the pump 32 may be powered on, the inlet valve 34 opened, the outlet valve 36 adjusted to achieve a first target fluid pressure in the cavity 18, and power to the heater 40 adjusted to achieve a first target temperature. The first target fluid pressure and temperature may be imposed for the duration of the cycle. A cycle commonly lasts a period of time of between about 4 to 24 hours, although shorter or longer cycle times may also be used. To complete the cycle, fluid pressure may be returned to approximately ambient pressure by opening the outlet valve 36, and turning off the pump 32 and/or closing the inlet valve 34. The fluid temperature may also be returned to approximately ambient temperature and allowing the contents of the vessel 12 to cool. The force F on the piston 16 may also be released. Then, without removing the proppant sample 70 from the vessel 12, another testing cycle may begin. The crosshead 24 may again be moved to impose a crushing level of force on the proppant sample 70 by the piston 16. The pump 32 may be powered on again, and the inlet valve 34 opened to resume fluid flow to the vessel 12. The outlet valve 36 may be adjusted to impose an elevated fluid pressure, and the heater 40 may be controlled to produce an elevated temperature. The elevated fluid pressure and temperature selected for the second cycle may be the same as for the previous cycle, or different fluid pressure and/or temperature values may instead be selected than for the previous cycle. Additional cycles may subsequently be imposed without removing the proppant sample 70 from the vessel 12. After the desired number of testing cycles have been performed, the piston 16 may be removed from the vessel 12 so the proppant sample 70 can be retrieved for subsequent inspection and analysis.

FIG. 4 is a sectional view of a portion of the proppant sample 70 after one or more testing cycles. As will be evident in the subsequent analysis, some of the individual proppant particles, such as particles 71, 72, remain intact. Other particles are fragmented into multiple smaller particles along cracks 75. For example, particles 73A, 73B, and 73C are fragments of a formerly single particle that was present prior to performing the test. Although the crushing force provided by the piston 16 (see FIG. 4) is largely responsible for the particles being crushed and fragmented, the simultaneous imposition of heat and pressurized fluid flow during the testing cycle(s) may impose additional stresses or may degrade the proppant sample 50, which tends to increase the amount of crushing and fragmenting that occurs under the crushing force of the piston 16. These testing conditions and results thereof closely simulate some of the harsh downhole conditions during an actual fracturing operation.

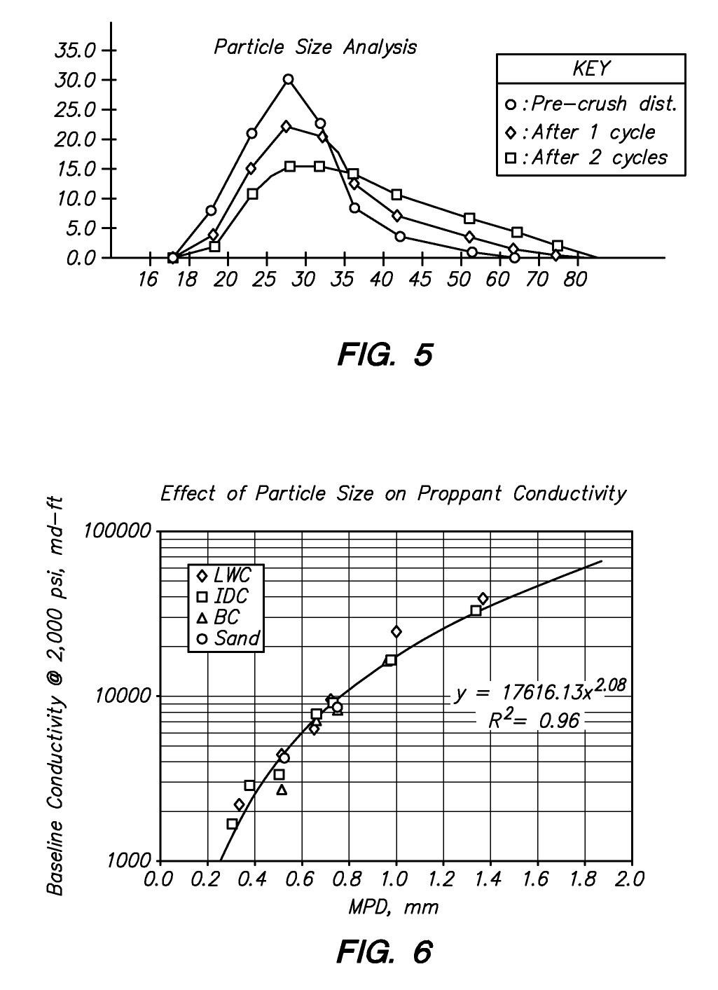

FIG. 5 is a graph of a particle size analysis performed on the proppant sample 70 both before and after the hot wet crush test is performed. The particle size analysis may be performed in any of a number of ways, such as by using sieves to separate difference sizes of particles, or an optical analyzer or LPSA to scan and detect the sizes of particles. The sieve analysis yields acceptable results using relatively inexpensive equipment, and can be performed in the field or in the laboratory. The optical analyzer or LPSA equipment is more expensive and better suited for a laboratory environment. The LPSA equipment is also capable of analyzing a wet or dry proppant sample. The horizontal axis of the graph is oriented in order of decreasing particle size, optionally expressed in terms of the standardized “US mesh” scale. The term “mesh” is derived from the mesh material that may be used, such as in a sieve, to determine the particle size distribution of a particulate material. For example, a “16” mesh on this scale denotes the smallest particles that would be retained by a mesh having 16 lines per inch. The vertical axis indicates the percentage of particles retained at a particular mesh. Thus, for example, 0% of the proppant sample 70 will be retained by 16 mesh, which indicates that all of the particles in the proppant sample 70 are less than 16 mesh in diameter. By convention, the horizontal axis is expressed in terms of mesh size and the vertical axis is expressed in terms of the percentage of particles retained by a particular mesh size, even though the particle size distribution may be determined using an optical analyzer or LPSA instead of a mesh.

The graph in FIG. 5 plots three exemplary curves, as indicated in the graph key. The “pre-crush” curve plots the particle size distribution prior to performing the Hot Wet Crush test. The “1 cycle” curve plots the particle size distribution resulting from performing a one-cycle test on the proppant sample 70. The “2 cycle” curve plots the particle size distribution resulting from performing a two-cycle test on the proppant sample 70. The two cycle test involves performing two cycles on a proppant sample without removing the proppant sample from the test vessel. If a proppant sample is removed from the test vessel after one cycle to perform a particle size analysis, that particular proppant sample is typically not returned to the test vessel for an additional cycle, because small fines may be lost. Rather, if both a single-cycle test and a multi-cycle test are to be performed, then two separate but equivalent proppant samples from the same source are used.

As the particle size analysis graph in FIG. 5 illustrates, each additional cycle generally shifts the resulting plot of the particle size distribution downward and to the right. Thus, the “1 cycle curve is lower than, wider than, and shifted to the right of the curve for the pre-crush distribution, and the curve for the two-cycle distribution is lower than, wider than, and shifted to the right of the curve for the one-cycle distribution. This tendency is due to the crushing and resulting fragmentation of particles that occurs under a crushing force, particularly when that level of force is combined with conditions of elevated pressures, temperature, and fluid flow.

While the testing methods described herein are different than a conventional conductivity test, one implication of the changing particle size is a change in the conductivity of the proppant. The particle size distribution of a proppant directly affects the conductivity of the proppant, which directly affects the permeability of the fractured well, i.e. the amount of oil and gas that can be recovered in economic quantities from a fractured well. Accordingly, the significance of the change in particle size distribution is that the decreasing particle size generally tends to decrease the permeability of the fractured well. The fines that develop from a proppant being exposed to downhole conditions tend to clog the fissures that result from fracturing a well, greatly decreasing the conductivity of the proppant and correspondingly decreasing the permeability of the well. FIG. 6 is a graph illustrating the correlation between particle size, in terms of the median particle diameter (“MPD”), and baseline conductivity of a proppant at two-thousand psi for each of four different proppant materials. The four proppant materials are lightweight ceramic (“LWC”), intermediate density ceramic (“IDC”), bauxite ceramic (“BC”), and sand. The graph shows a clear trend that conductivity increases with increasing MPD. Stated differently, the graph shows how conductivity decreases with decreasing particle size, for all four proppant materials. Thus, it is important to know how downhole conditions decrease particle size.

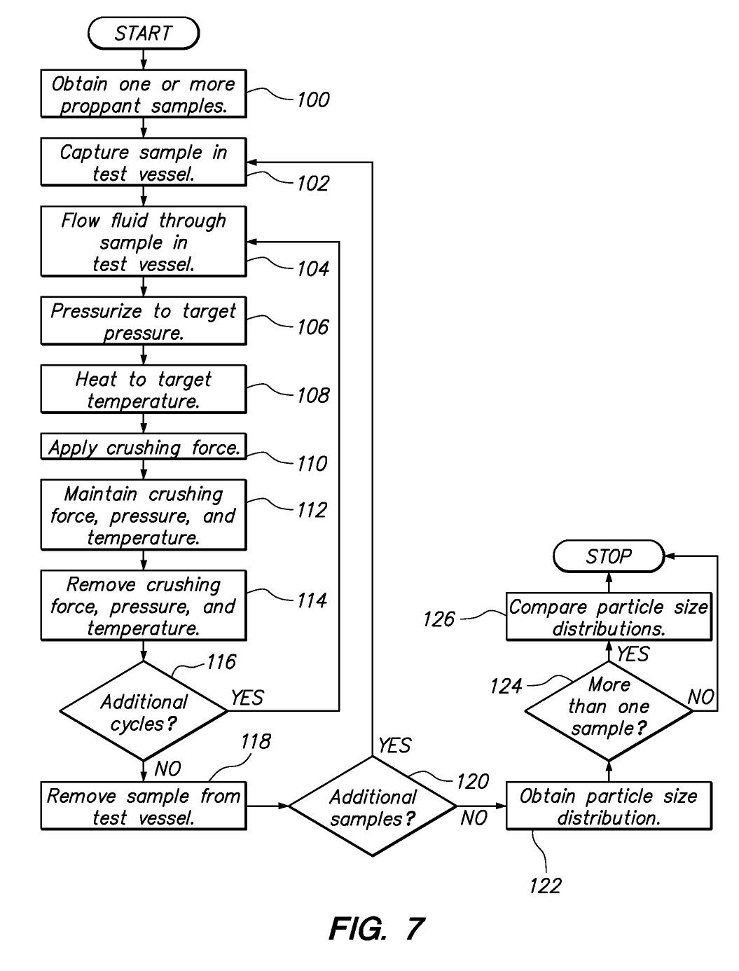

FIGS. 1-6 and the discussion thereof pictorially illustrate and describe an apparatus and testing method according to an embodiment of the invention. FIG. 7 is a flowchart outlining a testing method according to an embodiment of the invention. One or more proppant samples are obtained in step 100. One of the proppant samples is selected and captured in a test vessel in step 102. Fluid flow is established through the proppant sample in step 104. The contents of the test vessel, including the proppant sample and fluid flowing through the proppant sample, are pressurized to a target pressure greater than atmospheric pressure in step 106 and heated to a temperature of more than ambient temperature in step 108. A crushing level of force is applied to the proppant sample in step 110. According to step 112, the crushing force, pressure, and temperature are maintained for a period of time. During this time, the crushing force, pressure, and temperature may be dynamically adjusted, such as by optionally varying the pressure over a target pressure range and optionally varying the temperature over a target temperature range. Typically, the temperature and pressure will be increased simultaneously while the crushing force is maintained. The crushing force, pressure, and temperature are removed in step 114. The force may be reduced to substantially zero, the pressure may be reduced to about ambient pressure, and the contents of the test vessel may be allowed to cool to about ambient temperature before performing another cycle (if at all) according to conditional step 116. In step 118, after the desired number of one or more testing cycles, the proppant sample is removed from the test vessel. One or more additional proppant samples may be selected and tested according to conditional step 120. After the desired number of proppant samples have been tested, the particle size distribution for the proppant samples may be obtained in step 122. If more than one proppant sample was tested, as queried in step 124, the particle size distributions may be compared in step 126. Additional analysis may also be performed, such as by computing an expected conductivity of the tested proppant samples from a predetermined correlation between conductivity and particles size, such as provided in FIG. 6.

The apparatus and testing methods provided under the embodiments of the invention described herein are designed to give a more realistic indication of the performance of a particular proppant and the resulting permeability of a fractured well over time. The effect of placing a crushing force, combined with temperature, fluid pressure, and fluid flow on a proppant in the laboratory better replicate the actual conditions a proppant will experience in service. The results of the testing and the accompanying particle size and conductivity analyses provide a well operator a better indication of the performance of a particular proppant in the well. Accordingly, the well operator may make a more informed choice when selecting a suitable proppant material in order to maximize the recovery of oil and gas from the well.

As will be appreciated by one skilled in the art, the present invention may be embodied as a system, method, or computer program product. Accordingly, the present invention may take the form of an entirely hardware embodiment, an entirely software embodiment, or an embodiment combining software and hardware aspects. Furthermore, embodiments of the present invention may take the form of a computer program product embodied in any tangible medium of expression having computer usable program code embodied in the medium.

Any combination of one or more computer usable or computer readable medium(s) may be utilized. The computer-usable or computer-readable medium may be, for example but not limited to, an electronic, magnetic, optical, electromagnetic, infrared, or semiconductor system, apparatus or device. More specific examples of the computer-readable medium include any of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CDROM), an optical storage device, or a magnetic storage device. The computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, or RF.

Computer program code for carrying out operations of the present invention may be written in any combination of one or more programming languages, including an object oriented programming language such as Java, Smalltalk, C++ or the like and conventional procedural programming languages, such as the “C” programming language or similar programming languages. With reference to the hardware of FIGS. 1-6, some of the program code may execute on the computer 48, and other program code may execute on any of the various controllers in communication with the computer 48, such as the hydraulic press controller 15, a controller for the pump 32, a controller for the inlet valve 34, and a controller of the back-pressure regulator 33.

With reference to FIG. 7, an embodiment of the invention may include computer program code for satisfying the exemplary method outlined in the flowchart. It will be understood that each block of the flowchart illustrations and/or block diagrams, and combinations of blocks in the flowchart illustrations and/or block diagrams, or functions/acts described with reference to system or apparatus figures, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, implement the functions/acts specified in the flowchart and/or block diagram block or blocks, or functions/acts described with reference to system or apparatus figures.

These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart, block diagram blocks, or with respect to the apparatus or systems shown. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart, specified in the block diagram blocks, and/or specified with reference to the system or apparatus shown in the figures.

Any flowchart and block diagrams in the figures may illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The description of the present invention has been presented for purposes of illustration and description, but it not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method of testing a particulate material, comprising:
capturing a sample of particulate material in the cavity of a test vessel between a cavity wall and a piston sealed with the cavity wall;
heating the sample of particulate material to a target temperature greater than ambient temperature;
flowing a fluid through the sample of particulate material from a fluid inlet of the test vessel to a fluid outlet of the test vessel;
pressurizing the fluid flowing through the sample to a target fluid pressure greater than ambient pressure; and
moving the piston within the cavity into direct contact with the particulate material with a target level of force sufficient to crush at least a portion of the particulate material while maintaining one or both of the temperature and the fluid pressure for one or more test cycles.

2. The method of claim 1, further comprising:
removing the sample of particulate material from the test vessel after one or more test cycles; and determining the particle size distribution of the sample.

3. The method of claim 2, wherein the step of determining the particle size distribution of the sample of particulate material comprises passing the sample through one or more sieves.

4. The method of claim 2, wherein the step of determining the particle size distribution of the sample of particulate material comprises performing one or both of an optical particle size analysis and a laser particle size analysis on the sample.

5. The method of claim 2, further comprising performing the step of determining the particle size distribution of the sample of particulate material while the sample is still wet from the fluid.

6. The method of claim 2, further comprising estimating the permeability of a proppant material having the determined particle size distribution by comparing the determined particle size distribution of the sample with a pre-determined correlation of particle size and permeability.

7. The method of claim 1, wherein the step of pressuring the fluid flowing through the sample comprises generating a back pressure to the fluid outlet of the test vessel.

8. The method of claim 1, further comprising:
dynamically varying the fluid pressure between a lower pressure of at least 10 pounds per square inch and an upper pressure of up to 20,000 pounds per square inch during one or more of the cycles.

9. The method of claim 1, wherein the fluid is water.

10. The method of claim 1, wherein the fluid includes a hydrocarbon selected from the group consisting of a brine, a hydrocarbon gas, a hydrocarbon liquid, and a hydrocarbon condensate.

11. The method of claim 1, wherein the particulate material is selected from the group consisting of ceramic particles, sand, glass beads, treated or resin-coated nut shells, metal shot, and metallic particles.

12. The method of claim 1, wherein each test cycle has a duration of at least two minutes.

13. A computer program product comprising a computer usable medium including computer usable program code for testing a sample of particulate material captured in a test vessel between a cavity wall and a piston sealed with the cavity wall, the computer program product including:
computer usable program code for controlling a heater to heat the sample to a target temperature greater than ambient temperature;
computer usable program code for controlling one or more valves to flow fluid into the test vessel;
computer usable program code for controlling a back-pressure regulator to pressurize the test vessel to a target pressure greater than ambient temperature; and
computer usable program code for controlling movement of the piston into direct contact with the particulate material with sufficient force to crush at least a portion of the particulate material while maintaining one or both of the temperature and the fluid pressure for one or more test cycles, each test cycle having a duration of at least about two minutes.

14. The computer program product of claim 13, further comprising computer usable program code for controlling the one or more valves and the back-pressure regulator to dynamically vary the fluid pressure between a lower pressure of at least 10 pounds per square inch and an upper pressure of up to 20,000 pounds per square inch during one or more of the cycles.

15. The computer program product of claim 13, further comprising computer usable program code for heating the fluid to a temperature of between 200 and 450 degrees Fahrenheit.

16. A system for testing a particulate material, comprising:
a test vessel having a cavity and a piston removably disposed within the cavity and sealed with the cavity wall, the cavity being sized for receiving a quantity of particulate material between the piston and the cavity wall;
a crosshead coupled to the piston and configured for moving the piston;
a heater in thermal contact with the test vessel;
a fluid system including a fluid source in fluid communication with an inlet port of the test vessel, a pump configured for pumping fluid from the fluid source to the test vessel, and a back-pressure regulator in fluid communication with an outlet port of the test vessel; and
one or more controllers configured for controlling the crosshead to move the piston into direct contact with the particulate material with a target force sufficient to crush at least a portion of the particulate material, for controlling the pump to pump fluid from the fluid source to the test vessel, for controlling the heater to heat the fluid in the test vessel to a target temperature above ambient temperature, and for controlling the back-pressure regulator to induce a target pressure greater than ambient pressure for a period of time.

17. The system of claim 16, further comprising:
a computer system in electronic communication with the one or more controllers and having a user interface configured for providing target test parameters including one or more of the target force, the target temperature, and the target pressure to the one or more controllers.

18. The system of claim 17, further comprising one or both of a pressure transducer configured for detecting the fluid pressure and a temperature sensor configured for detecting fluid temperature, wherein the computer system is in electronic communication with the pressure transducer and the temperature sensor and the user interface is configured for displaying the detected fluid pressure and the detected fluid temperature.

19. The system of claim 17, wherein the computer system is configured for automatically conducting a plurality of test cycles, wherein each test cycle comprises a target temperature, pressure, and crushing force for a period of at least 120 seconds.

20. The system of claim 17, wherein the computer system is configured for heating the fluid to a temperature of between 200 and 450 degrees Fahrenheit
